(19) United States
(12) Patent Application Publication
(10) Pub. No.: US 2009/0090439 A1
(43) Pub. Date: Apr. 9, 2009

(54) COLD WORKING LUBRICANT AND COLD WORKING METHOD FOR STEEL PIPE

(75) Inventors: MASAYOSHI AKIYAMA, Kobe-shi (JP); Tsutomu Arita, Wakayama-shi (JP)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: SUMITOMO METAL INDUSTRIES, LTD., Osaka-shi (JP)

(21) Appl. No.: 12/270,595

(22) Filed: Nov. 13, 2008

Related U.S. Application Data
(63) Continuation of application No. PCT/JP2007/059966, filed on May 15, 2007.

(30) Foreign Application Priority Data
May 15, 2006 (JP) 2006-135154

(51) Int. Cl. C21D 7/02; C07C 53/126

(52) U.S. Cl. 148/591; 554/1

(57) ABSTRACT

The present invention provides a lubricant comprising an alkali soap and a cold working method, in which the lubricant layer can be easily formed on the surface of a steel pipe prior to cold working the reduces the work load during cold working of the steel pipe, and whose layer can be easily removed by washing the surface of the steel pipe after cold working. After that, an alkali soap aqueous solution or aqueous pasty alkali soap is applied to the working surface of the steel pipe, cold working of the steel pipe is performed, and thereafter the alkali soap layer is removed by washing with water. The alkali soap aqueous solution or aqueous pasty alkali soap is preferably prepared within a vessel having an inner surface consisting of a non-metal material.

### FIELD OF THE INVENTION

The present invention relates to a cold working lubricant and a cold working method for steel pipe. More specifically, the present invention relates to a lubricant excellent in lubricating property during cold working of a steel pipe and excellent in washing removability from the steel pipe surface after cold working, and a cold working method for steel pipe using the same.

### BACKGROUND OF THE INVENTION

In cold working of a steel pipe, lubricating treatment has been executed for the purpose of reducing a working load and preventing a seizure between the steel pipe and a working tool.

Three methods have been conventionally known for the lubricating treatment in cold working of a metal material including the steel pipe, namely, a chemical treatment method, an oil lubricating method and a synthetic resin layer lubricating method.

The chemical treatment method comprises a chemical treatment process such as pickling for removing an oxide layer or a hydroxide layer formed on a surface of a metal material, and a layer process for forming a substrate layer such as a phosphate layer or an oxalate layer, followed by a process for forming a metal soap layer including a non-alkali metal such as Zn on the substrate layer. The substrate layer and the metal soap layer are formed on a work piece surface through these processes. The layer formed by the chemical treatment has an excellent lubricating property. The chemical treatment method is frequently used for a pretreatment, mainly for the cold working of a steel wire rod or a steel bar.

The oil lubricating method comprises coating lubricating oil such as mineral oil to a working surface of metal material. The cold working is performed after coating the lubricating oil. The oil lubricating method among the lubricating treatment methods is extensively used for cold working because the lubricating oil that forms a lubricant layer can be easily coated. The oil lubricating method is applied mainly to pipe expansion work, diameter reducing work, cold drawing work, cold rolling and the like of steel pipe.

The synthetic resin layer lubricating method comprises forming a synthetic resin layer on a working surface. This synthetic resin layer functions as a lubricant during cold working. The synthetic resin layer lubricating method is applied mainly to press working of the steel sheet or the like.

However, all these lubricating treatment methods have problems as described below. Particularly, the application to lubricating treatments in the cold working of a steel pipe is problematic.

The chemical treatment method cannot be adopted except for cold working of a steel wire rod or a steel bar, because it includes many processes for forming a substrate layer and thus requires large-scaled facilities and troublesome works. In the synthetic resin layer lubricating method, in order to prevent peeling of the synthetic resin layer during cold working, the synthetic resin layer is needed to be firmly adhered to the surface of the metal material, which results in an increased cost caused by an enlarged scale of facilities and a troublesome work. Therefore, this method would not be adopted except for cold working of a steel sheet.

On the other hand, the oil lubricating method requires neither a troublesome work nor an enlarged scale of facilities, compared with the chemical treatment method and the synthetic resin layer lubricating method. However, the oil lubricating method does not reduce the working load more than the chemical treatment method or the synthetic resin layer lubricating method. In the oil lubricating method, because the lubricating oil is simply coated to the working surface of the metal material, the lubricating oil such as mineral oil that is applied to the surface of the metal material is low in the adhesiveness, and may not adhere to the part of the surface of the metal material. It results in seizing on this part.

In all theses lubricating treatment methods, it is difficult to remove the lubricant or the lubricating oil from the surface of the metal material after cold working. Accordingly, some lubricant or lubricating oil is apt to remain on the surface of the resulting metal product after removal treatment thereof. The remaining lubricant or lubricating oil may cause various problems in a heat treatment process or the like after the cold working.

When a metal material with a chemical treatment layer consisting of a phosphate remaining on the surface is heat-treated, for example, a phosphorization to the metal material may deteriorate the material strength. Remaining lubricating oil on stainless steel material, consisting of mineral oil, causes carburization to the stainless steel product during heat treatment. When a metal soap layer containing a non-alkali metal salt of Zn, Mn or the like remains on the surface, the same problem is caused during heat treatment. Namely, the lubricant remaining on the surface may deteriorate the mechanical characteristics of the surface of the metal product during heat treatment. Further, since the lubricating oil or synthetic resin layer is regarded as dirt, the product with remaining on the surface cannot be sold thereafter. Because of this problem, the lubricant layer or lubricating oil formed by the lubricating treatment must be removed after cold working. It is preferable that the lubricant or lubricating oil for cold working of a metal material can be easily removed from the surface of the metal material after cold working, in addition to the excellent lubricating property in the cold working of the metal material.

Besides the three lubricating treatment methods described above, a method, which is related to the press working of an aluminum plate, is disclosed in Patent document 1. This method comprises a liquid lubricant consisting of a mixture of fine lubricant particles such as molybdenum disulfide and graphite with metal soap applied to the working surface of an aluminum plate, prior to press working However, this method aims at press working of a sheet metal such as the aluminum plate with extremely low cold deformation resistance, and is scarcely applicable to the lubricating treatment for cold working of a pipe-shaped metal, which involves harsh plastic deformation in addition to high cold deformation resistance, such as a cold working of steel pipe including pipe expansion work or cold drawing work. Furthermore, the lubricant disclosed in Patent document 1 is difficult to be removed, and when it is applied to the lubricating treatment during the cold working of a steel pipe, particularly, the fine lubricant particles such as molybdenum disulfide and graphite are scarcely removed from the steel pipe surface. Because, when the oxide or a hydroxide layer is formed on the surface of the steel pipe, a minute unevenness or crack is apt to occur on the oxide or hydroxide layer, which would trap fine particles of the lubricant such as molybdenum disulfide and graphite cannot be removed.

In relation to the working of the aluminum plate, a solid lubricant method is disclosed in Patent Document 2. This method requires a solid lubricant consisting of 3-18% surfactant, 0.03-4.0 wt % rust preventive agent, and the balance of a water-soluble or water-dispersible film forming component such as an α-olefin/maleic monoester/maleic acid monoester salt terpolymer that is a polymeric synthetic wax having a molecular weight of 6000 or more, a carboxylated organic polymer compound having a molecular weight of 1000 or more and a salt thereof. However, this costly solid lubricant, although used for warm press working of a sheet metal such as an aluminum plate with an extremely low cold deformation resistance, cannot be applied to the lubricating treatment for cold working of a pipe-shaped metal such as pipe expansion work or cold drawing work with a high cold deformation resistance, which requires a harsh plastic deformation.

Patent Document 1: Japanese Patent Unexamined Publication No. H6-277766.

Patent Document 2: Japanese Patent Unexamined Publication No. 6-264086

### DISCLOSURE OF THE INVENTION

### Problems to be Solved by the Invention

Due to these circumstances, one objective of the present invention is to provide a cold working lubricant for a steel pipe, which much reduces the work load on the cold working of a steel pipe, which can easily form a lubricant layer on the surface of the steel pipe prior to cold working, and is excellent in washing removability thereof from the steel pipe surface after cold working.

Another objective of the present invention is to provide a cold working method for the steel pipe using this lubricant.

### Means for Solving the Problems

As a result of examinations and experiments for various lubricants from the viewpoint of an easy formation of a lubricant layer onto a surface of a steel pipe prior to cold working and easy removal, the present inventors obtained the following knowledge, focusing in alkali soap.

The alkali soap means a water-soluble alkali metal salt (Na salt or K salt) of long-chain fatty acid. The alkali soap can be easily coated to the working surface of a steel pipe by making it into an alkali soap aqueous solution because it is water-soluble. The layer formed on the working surface of the steel pipe exists as a lubricant layer on the surface of the steel pipe as it is or in a dried state, while the lubricant layer after cold working can be easily removed by washing the surface of the steel pipe with water or hot water after cold working, because it forms the water-soluble alkali soap.

Otherwise, instead of the alkali soap in the state of an alkali soap aqueous solution, an aqueous pasty alkali soap that has some flowability can be coated to the working surface of the steel pipe. Impregnating the alkali soap with water makes this aqueous pasty alkali soap. This is, due to the pasty, conveniently used when the coating is performed to only a part of the working place that requires a lubricant layer. The aqueous pasty alkali soap layer after drying is the same as the alkali soap aqueous solution layer after drying.

The alkali soap is a water-soluble alkali metal salt (Na salt or K salt) of long-chain fatty acid as described above, and any straight chain fatty acid is adoptable thereto regardless of whether the saturated fatty acid or unsaturated fatty acid. Preferably, the alkali soap is composed of either or both of Na salt and K salt of one or more kinds of straight-chain fatty acids having 10 to 18 carbon atoms. Specific examples thereof include such as capric acid (C₉H₁₉COOH), lauric acid (C₁₁H₂₃COOH), myristic acid (C₁₃H₂₇COOH), palmitic acid (C₁₅H₃₁COOH), palmitoleic acid (C₁₅H₂₉COOH), margalinic acid (C₁₆H₃₃COOH), stearic acid (C₁₇H₃₅COOH), oleic acid (C₁₇H₃₃COOH), and linoleic acid (C₁₇H₃₁COOH).

With respect to coating the alkali soaps having various chemical compositions on a surface of a steel pipe, lubricating property and washing removability thereof were examined. The result is shown blow.

The alkali soaps, having chemical compositions shown in Table 1, were prepared.

[Table 1]

TABLE 1

| | Sample | Sample | Sample | Sample | Sample | Sample |
|---|---|---|---|---|---|---|
| Alkali soap | No. 1 | No. 2 | No. 3 | No. 4 | No. 5 | No. 6 |
| Na caprate | — | 3 | — | — | — | — |
| Na laurate | 30 | 23 | — | — | — | — |
| Na myristirate | 10 | 35 | 2 | — | — | 2 |
| Na palmitate | 30 | 38 | 38 | 2 | 5 | 18 |
| Na palmitoleate | — | — | 4 | 7 | 6 | 4 |
| Na stearate | — | 1 | 15 | — | 7 | 35 |
| Na oleate | 30 | — | 37 | 75 | 68 | 37 |
| Na linorate | — | — | 4 | 16 | 14 | 4 |

For each of the alkali soap layers, a pendulum friction test was performed and the lubricating property concerned was evaluated by measuring the frictional coefficient. The test conditions are as follows.

Each of various alkali soaps having chemical compositions shown in Table 1 was dissolved in water to prepare an alkali soap aqueous solution with concentration of 11 mass %. A specimen ball was covered with this aqueous solution and dried with cold air to form a layer, and a frictional coefficient (μ) thereof was measured. The measurements were 30 times performed for each specimen at a room temperature (25° C.). Table 2 shows the friction coefficient in the first measurement and the friction coefficient as a stabilized value for each sample. For a sample whose friction coefficient exceeded 0.3μ before the final measurement, the number of times of measurement until the friction coefficient exceeded 0.3μ was shown.

[Table 2]

TABLE 2

| | Friction | Friction | Number of times of |
|---|---|---|---|
| | coefficient | coefficient | measurement |
| | (μ) in 1^{st} | (μ) as stabilized | until the friction |
| | measurement | value | coefficient exceeded 0.3μ |
| Sample No. 1 | 0.089 | 0.448 | 11 |
| Sample No. 2 | 0.158 | 0.387 | 19 |
| Sample No. 3 | 0.097 | 0.100 | — |
| Sample No. 4 | 0.101 | 0.104 | — |
| Sample No. 5 | 0.110 | 0.300 | 13 |
| Sample No. 6 | 0.102 | 0.105 | — |

Regarding the washing removability of each layer, a specimen having each layer in a dried state was washed in water with slightly stirring, and the adhesion amount of the layer was measured before and after washing, whereby the degree of washing removal was evaluated. The forming condition and test condition of the specimen are as follows.

Each of various alkali soaps having chemical compositions shown in Table 1 was dissolved in water to prepare an alkali soap aqueous solution with concentration of 11 mass %. This aqueous solution was coated by spraying onto one side of a SUS thin plate specimen (80 mm×60 mmxlmm) with a layer thickness of about 30 g/m² (in dried state), followed by drying for 24 hours by use of a dryer of 50° C., whereby a dry layer was formed on the specimen. The specimen with the dry layer was dunked in a water bath (1000-mL beaker) of 50° C. under stirring (just to whirl), and the time (sec) for the removal of the layer by washing was measured. The washing removal time of each specimen is shown in Table 3.

[Table 3]

TABLE 3

| | Washing time (sec) |
|---|---|
| Sample No. 1 | 10-15 |
| Sample No. 2 | 15-20 |
| Sample No. 3 | 30-40 |
| Sample No. 4 | 20-30 |
| Sample No. 5 | 20-25 |
| Sample No. 6 | 40-50 |

Consequently, it was found that a layer can be easily formed on a working surface by coating water-soluble alkali soap thereto, and the resulting layer is excellent in lubricating properties with a low friction coefficient. Further, when such a layer is formed, it is also easy to remove the layer by washing after cold working.

The present invention has been achieved based on the new knowledge above. The cold working lubricants for a steel pipe according to the present invention are shown in the following (1) to (3). The cold working methods for a steel pipe according to the present invention are shown in the following (4) to (12). Hereinafter each one will be referred to as the present invention (1) to (12), respectively. These may collectively be referred to as the present invention.

(1) A cold working lubricant for a steel pipe, comprising alkali soap.

(2) The cold working lubricant for a steel pipe according to (1) above, wherein the cold working of a steel pipe is a pipe expansion work of a steel pipe end using a plug.

(3) The cold working lubricant for a steel pipe according to (1) or (2) above, wherein the alkali soap is composed of either or both of Na salt and K salt of one or more kinds of straight-chain fatty acids having 10 to 18 carbon atoms.

(4) A cold working method for a steel pipe, comprising cold working after forming a solid alkali soap layer on the working surface of a steel pipe by coating an alkali soap aqueous solution thereto.

(5) A cold working method for a steel pipe, comprising cold working after forming a solid alkali soap layer on the working surface of a steel pipe by coating an alkali soap aqueous solution thereto followed by drying.

(6) The cold working method for a steel pipe according to (4) or (5), wherein the alkali soap aqueous solution to be coated to the working surface of the steel pipe is prepared by dissolving alkali soap in water within a vessel having an inner surface consisting of a non-metal material.

(7) A cold working method for a steel pipe, comprising cold working after forming a solid alkali soap layer on the working surface of a steel pipe by coating an aqueous pasty alkali soap thereto.

(8) A cold working method for a steel pipe, comprising cold working after forming a solid alkali soap layer on the working surface of a steel pipe by coating an aqueous pasty alkali soap thereto followed by drying.

(9) The cold working method for a steel pipe according to (7) or (8), wherein the aqueous pasty alkali soap to be coated to the working surface of the steel pipe is prepared by impregnating alkali soap with water within a vessel having an inner surface consisting of a non-metal material.

(10) The cold working method for a steel pipe according to any one of (4) to (9) above, wherein the solid alkali soap layer on the working surface is removed after cold working of the steel pipe, by washing the working surface with water or hot water.

(11) The cold working method for a steel pipe according to any one of (4) to (10) above, wherein the cold working of the steel pipe is a pipe expansion work of the steel pipe end using a plug.

(12) The cold working method for a steel pipe according to any one of (4) to (11) above, wherein the alkali soap is composed of either or both of Na salt and K salt of one or more kinds of straight-chain fatty acids having 10 to 18 carbon atoms.

The alkali soap referred to herein means alkali metal salt (Na salt or K salt) of water-soluble long-chain fatty acid as described above. Any straight-chain fatty acid can be used thereto regardless of whether a saturated fatty acid or an unsaturated fatty acid. Particularly, the alkali soap is preferably composed of either or both of Na salt and K salt of one or more kinds of straight-chain fatty acids having 10 to 18 carbon atoms. Specifically, Na or K salts of capric acid (C₉H₁₉COOH), lauric acid (C₁₁H₂₃COOH), myristic acid (C₁₃H₂₇COOH), palmitic acid (C₁₅H₃₁COOH), palmitoleic acid (C₁₅H₂₉COOH), margalinic acid (C₁₆H₃₃COOH), stearic acid (C₁₇H₃₅COOH), oleic acid (C₁₇H₃₃COOH), and linoleic acid (C₁₇H₃₁COOH) are preferably used. The alkali soap such as the Na salt and K salt of the water-soluble long-chain fatty acid can be used independently or in combination as well. The cold working lubricant such as alkali metal salts of straight-chain fatty acid having 10 to 18 carbon atoms of the alkali soaps are preferably used, and the alkali metal salts of straight-chain fatty acid can be used independently or in combination of two or more kinds thereof.

The cold working lubricant such as the alkali soap may be coated to the surface of a working tool, but it is preferably coated to the working surface of the steel pipe. The cold working can be performed as the layer formed to the working surface of the steel pipe or to the wet surface of the working tool, or after drying it.

The steel pipe for cold working includes a stainless steel pipe. The steel pipe can be not only a seamless steel pipe manufactured by Mannesmann process or Ugine-Sejournet process, but also a hot-forged steel pipe or a welded steel pipe.

The cold working method includes pipe expansion work of a steel pipe end using a plug and drawing work of the steel pipe.

In one cold working method according to the present invention, an alkali soap aqueous solution is used for a lubricant layer by coating it to the working surface of a metal material that is not subjected to substrate treatment in order to form a solid alkali soap layer thereon. Although the cold working may be performed as it is, the lubricant layer is preferably dried prior to the cold working. Thus, the lubricant layer can be easily formed without executing a substrate treatment process in the chemical treatment. Further, the lubricating treatment method by an alkali soap layer reduces workload more than the oil lubricating method or synthetic resin layer lubricating method. The working surface of the steel pipe may be in a surface-exposed state by executing descaling by shot blasting or pickling after shaping the metal material by rolling or the like, or in a state remaining on the surface after rolling with a scale layer that is an oxide or with a rust layer that is a hydroxide.

The alkali soap aqueous solution to be coated to the working surface of the steel pipe is preferably prepared by dissolving the alkali soap in water within a vessel that has an inner surface consisting of a non-metal material. The non-metal material includes, for example, resin, glass, and ceramics. Instead that the vessel itself is made of a non-metal material, only the inner surface of the vessel may be lined with or coated with the non-metal material. When the alkali soap is dissolved in water within a vessel whose inner surface is in contact with the alkali soap aqueous solution and consists of a metal material such as zinc (Zn) or tin (Sn), the alkali soap aqueous solution becomes semi-solidified. This semi-solidified alkali soap aqueous solution has the property of scarcely adhering to the working surface of the steel pipe. Therefore, it is difficult to coat the working surface with the alkali soap in a uniform thickness, and even if it is dried, a layer all over the whole working surface is scarcely formed. Consequently, the lubricating characteristic is deteriorated, and a seizure may be caused during working on the surface having no layer. The alkali soap aqueous solution should be prepared within the vessel whose inner surface is covered with a non-metal material, whereby the semi-solidification of the alkali soap aqueous solution can be prevented, and the adhesiveness of the alkali soap aqueous solution to the working surface is extremely enhanced. Consequently, the alkali soap can be uniformly coated to the working surface, and after drying it, a uniform solid alkali soap layer can be formed over the whole working surface.

In another cold working method according to the present invention, aqueous pasty alkali soap is coated to a working surface of a metal material not subjected to substrate treatment in order to form a solid alkali soap layer, whereby it is used as the lubricant layer. Although the cold working may be performed as it is, the lubricant layer is preferably dried after the coating prior to the cold working. Thus, the lubricating layer can be easily formed without executing a substrate treatment process in chemical treatment. Further, the lubricating treatment method by alkali soap layer shows better load reducing effect than the oil lubricating method or synthetic resin layer lubricating method. The working surface of the steel pipe may be in a surface-exposed state by executing descaling by shot blasting or pickling after shaping the metal material by rolling or the like, or in a state remaining on the surface after rolling with scale layer that is an oxide or with rust layer that is a hydroxide.

The aqueous pasty alkali soap can be prepared by impregnating the alkali soap with warm water and cooling it to room temperature, then into a pasty state, while maintaining the softness to some degree. The preferable temperature of the warm water used for the preparation of the aqueous pasty alkali soap is 60° C. or higher. The aqueous pasty alkali soap to be coated to the working surface of the steel pipe is preferably obtained by impregnating the alkali soap with water within a vessel that has an inner surface consisting of a non-metal material. The non-metal material includes, for example, resin, glass and ceramics. Instead that the vessel is entirely formed of the non-metal material, only the inner surface of the vessel may be lined with or coated with the non-metal material.

When the inner surface is in contact with the aqueous pasty alkali soap and consists of a metal material such as zinc (Zn) or tin (Sn), the aqueous pasty alkali soap has the property of scarcely adhering to the working surface of the steel pipe. Therefore, it is difficult to coat the working surface with the alkali soap in a uniform thickness, and even if it is dried, a layer all over the working surface is scarcely formed. Consequently, the lubricating characteristic is deteriorated, and seizure may be caused during working on the surface having no layer. The aqueous pasty alkali soap should be prepared within a vessel whose inner surface is coated with the non-metal material, whereby the adhesiveness of the aqueous pasty alkali soap to the working surface is extremely enhanced. Consequently, the alkali soap layer can be uniformly formed on the whole working surface.

In the present invention, the aqueous pasty alkali soap to be coated to the working surface of the steel pipe is preferably obtained by impregnating the alkali soap with water within a vessel having an inner surface consisting of a non-metal material. The non-metal material includes, for example, resin, glass and ceramics.

When the alkali soap is impregnated with water within a vessel whose inner surface in contact with the aqueous pasty alkali soap consisting of a metal material, for example, such as zinc (Zn) or tin (Sn), the aqueous pasty alkali soap is scarcely adhered to the working surface of the steel pipe. Naturally, the lubricating characteristic is deteriorated, and seizure is caused during working on the surface having no layer. The aqueous pasty alkali soap is prepared within the vessel whose inner surface is covered with the non-metal material, whereby the adhesiveness of the alkali soap aqueous solution to the working surface is extremely enhanced.

Since the alkali soap is easily dissolved in water, the working surface is washed with water or hot water after cold working, whereby the solid alkali soap layer remaining on the working surface can be easily removed. Consequently, the remaining lubricant layer can be suppressed or solved.

The steel pipe to which the cold working lubricant comprising alkali soap is coated includes a stainless steel pipe. The steel pipe can be not only a seamless steel pipe manufactured by Mannesmann process or Ugine-Sejournet process but also a hot-forged steel pipe or a welded steel pipe.

The cold working method for the steel pipe includes pipe expansion work of a steel pipe end using a plug, drawing work of steel pipe and the like.

### EFFECT OF THE INVENTION

According to the present invention, a layer of lubricant can be easily formed on the surface of a steel pipe prior to cold working with a high load reduction effect in cold working of the steel pipe, and the layer can be easily removed by washing of the steel pipe surface after cold working.

### BEST MODE FOR CARRYING OUT THE INVENTION

Embodiments of the present invention will be described in detail in reference to the attached drawings. In the drawings, descriptions for identical or corresponding parts are omitted by assigning the identical reference number thereto.

The cold working lubricant and cold working method for the steel pipe will be described below. The cold working of the steel pipe is a pipe expansion work of a steel pipe end using a plug.

In FIG. 1, an alkali soap aqueous solution of a lubricant is prepared (S1). Specifically, the alkali soap of Na salt and/or K salt of straight-chain fatty acid are prepared. A preferable main component of the alkali soap is Na stearate. The content of the Na stearate in the alkali soap may be such that the effect of the present invention can be shown. Preferably, the alkali soap contains 95 mass % or more of Na stearate.

The above-mentioned alkali soap is dissolved in water within a vessel that has an inner surface coated with a non-metal material in order to prepare the alkali soap aqueous solution. The non-metal material means, for example, resin such as plastics, glass or ceramics. When the alkali soap is dissolved in water within a vessel that has an inner surface consisting of a metal material such as a metal vessel, the alkali soap aqueous solution becomes semi-solidified. Such an alkali soap aqueous solution scarcely adheres to the working surface (inner surface or outer surface) of a steel pipe, and even if it could adhere to the working surface, the resulting layer is not uniform but uneven. Therefore, it is extremely difficult to uniformly coat the alkali soap onto the whole working surface. Although the cause of this is not necessarily certain, the following explanation should be considered. When solid alkali soap is dissolved in water within a vessel that has an inner surface consisting of a metal material, the metal element constituting the vessel inner surface is dissolved in the alkali soap aqueous solution. During this time, the dissolved metal element is bonded with long-chain fatty acid of the alkali soap to produce a metal soap (non-alkali metal salt of the long-chain fatty acid). This generation of metal soap causes considerably deterioration of the adhesiveness to the working surface.

Therefore, the alkali soap aqueous solution should be prepared within a non-metal vessel. The alkali soap aqueous solution can be uniformly adhered to the entire working surface with good adhesiveness. Increasing the amount of alkali soap added to the water results in increasing the viscosity of the alkali soap aqueous solution, and improves the adhesiveness to the working surface. When the alkali soap concentration in the alkali soap aqueous solution is set at 100 g/L (liter) to 450 g/L, the resulting alkali soap aqueous solution shows satisfactory adhesiveness. Even out of this concentrated range, the alkali metal soap aqueous solution is adhered to the entire working surface so that the effect of the present invention is displayed to certain degree.

The alkali soap aqueous solution prepared within the non-metal vessel is applied to the working surface that is not subjected to chemical treatment (S2). Specifically, the alkali soap aqueous solution is directly applied to an inner or outer surface of a steel pipe with a scale layer that is an oxide or rust layer, which is a hydroxide adhered thereto after rolling, or an inner or outer surface of a steel pipe free from scale or rust (or base metal surface) which is subjected to descaling or derusting treatment.

A chemical treatment layer formed by a chemical treatment (phosphate layer, oxalate layer, and metal soap layer) is scarcely removed after cold working since it is adhered to the steel pipe surface by chemical bonding. If the chemical treatment layer is left on the steel pipe inner or outer surface, mechanical characteristics of the steel pipe can deteriorate. For example, when a steel pipe's remaining zinc phosphate layer on the inner or outer surface is heat-treated or welded to another steel pipe, phosphorization can be caused and this may reduce the strength of the steel pipe. If oil of the oil lubrication method remains, the non-fitting of paint may cause a line pipe which connects steel pipes with the steel pipe inner or outer surface, from being painted. Therefore, in this situation, it is preferable to use a steel pipe that has not been subjected to chemical treatment or to oil lubrication.

The alkali soap solution is coated to a working surface of a steel pipe, for example, by the following methods. A worker such as an operator of a pipe expansion apparatus coats the alkali soap aqueous solution to the working surface by use of a brush or the like. Otherwise, the alkali soap aqueous solution may be coated to the working surface by dunking the steel pipe itself in the alkali soap aqueous solution within a non-metal vessel.

After the alkali soap aqueous solution is coated to the steel pipe's inner surface, the alkali soap aqueous solution is dried to form a solid alkali soap layer (S3). Since the alkali soap is applied closely to all of the working surface and results in a solid layer when dried, drying is preferably performed. The drying can be performed, for example, by use of a blower or the like for quick drying or by natural drying in the atmosphere.

After the solid alkali soap layer is formed, the resulting steel pipe is expanded (S4). At this time, the steel pipe whose inner surface has the solid alkali soap layer formed thereon is expanded in contact with a plug that is a working tool. The solid alkali soap layer has a higher adhesiveness to the working surface than the lubricating oil used in the conventional oil lubrication. Further, the oil escapes toward the lower pressure side when working pressure is applied because it is a fluid, resulting in deterioration of the lubricating performance. The solid alkali soap layer has poor flowability because it is solid and stays there even if the working pressure is applied. Therefore, the solid alkali soap layer can prevent direct contact to the steel pipe with the tool, which is more satisfactory in both lubricating property and seizure resistance, than in the oil lubrication. Consequently, flawing in the working surface can be prevented. Further, the lubricating by a solid alkali soap layer can reduce the workload more than the oil lubrication.

After the cold working, the working surface is washed with water to remove the solid alkali soap layer (S5). Since the alkali soap easily dissolves in water, the solid alkali soap layer adhered to the working surface can be easily removed by washing with water. Therefore, compared with the conventional lubricating treatment, the lubricant layer is mostly removed. Since the dissolution degree of alkali soap increases by raising the temperature of the water for washing, and even though the water temperature may be normal, the time necessary for removal can be also shortened. Namely, the alkali soap can be removed in a short time by washing with hot water.

In the cold working method according to the present invention, using alkali soap for the lubricant can easily form a lubricant layer. Therefore, the use of a plurality of different processes is not needed for forming the lubricant layer (chemical treatment layer) in comparison with the chemical treatment method, and is not needed for facilities for producing a substrate layer such as phosphate layer. The present invention reduces the workload more than the conventional oil lubrication or synthetic resin layer lubrication.

Further, the solid alkali soap layer that is the lubricant layer in the present invention can be easily removed by washing with water. Therefore, the lubricant layer can be removed more easily than in the conventional lubricating treatments (chemical treatment, oil lubrication and synthetic resin layer lubrication), and the remaining lubricant layer on the working surface of a metal product can be considerably removed.

Comparing the lubricant layers (chemical treatment layer, lubricating oil and synthetic resin layers) formed in the conventional lubricating treatments, the lubricant layer coated by the alkali soap in the present invention has a small environmental problem. Also the detergent used for removing the chemical treatment layer or lubricating oil has not only a large environmental problem, but also harmfully influences the human body. The lubricant layer according to the present invention can be easily removed with water, so that the environment and human body problems can be significantly reduced.

Instead of the alkali soap aqueous solution that is coated on the working surface mentioned above, the aqueous pasty alkali soap can be applied. Impregnating solid alkali soap with warm water and cooling to room temperature can be used to prepare the aqueous pasty alkali soap. The temperature of warm water is preferably 60° C. or higher and, more preferably, 80° C. or higher. The aqueous pasty alkali soap is preferably prepared within a vessel having an inner surface consisting of a non-metal material. The hardness of the aqueous pasty alkali soap is lower than general solid alkali soap, and substantially equal to, for example, the hardness of lipstick.

The aqueous pasty alkali soap prepared by the above-mentioned method is applied to a working surface of a steel pipe in the same manner as the alkali soap aqueous solution. The aqueous pasty alkali soap is solid having no flowability. Therefore, the aqueous pasty alkali soap can be easily applied to the working surface, particularly only to a place that requires a lubricant layer on the surface of the steel pipe. The aqueous pasty alkali soap is easy to adhere to the working surface because of it's low hardness, and thus can be easily uniformly coated.

The cold working is preferably carried out after drying the aqueous pasty alkali soap applied to the working surface.

Although the cold working is carried out at a normal temperature in the above-mentioned conditions, the present invention is applicable to hot working which is carried out by heating a steel pipe to a temperature of 150° C. or lower, which has the same effect as above.

### EXAMPLE 1

A seamless steel pipe was subjected to pipe expansion using Na stearate as a lubricant, and the load applied in the pipe expansion was examined.

A seamless steel pipe with a shape and a strength (grade) shown in Table 4 (hereinafter simply referred to as steel pipe) was prepared. In the table, the unit of outside diameter, inside diameter, pipe thickness and length are shown by mm, and the grade is based on the API standard. The material of the steel pipe is carbon steel.

[Table 4]

TABLE 4

| Shape | | | | |
|---|---|---|---|---|
| Outside | | | | |
| diameter | Inside diameter | Pipe thickness | Length | |
| (mm) | (mm) | (mm) | (mm) | Grade |
| 89.05 | 75.33 | 6.86 | 150 | 5CT3-P110 |

Three pipe expansion plugs 1 of a shape shown in FIG. 2 were prepared. A layer of 3 mm thickness was formed respectively on the surface 10 to contact with the inner surface of the steel pipe of each plug 1, using the materials and formation method shown in Table 5.

[Table 5]

TABLE 5

| Plug No. | Layer material |
|---|---|
| 1 | Cemented carbide |
| 2 | SKD steel |
| 3 | CrN layer by ion plating |

The plug of Plug No. 1 is a cemented carbide plug. The plug of Plug No. 2 is made of cold working tool steel (SKD steel). The plug layer of Plug No. 3 is CrN layer formed by ion plating. The maximum value of the plug diameter of each plug 1 is 76.8 mm.

The pipe expansion work was executed by use of an apparatus shown in FIG. 3 according to the following method. A steel pipe 2 was fixed between the plug 1 and a cylindrical pushing and pulling tool 4. After fixing, the pushing and pulling tool 4 was pushed by a press head 3 of a 150-t press machine arranged on the opposite side of the steel pipe 2 across the pushing and pulling tool 4, whereby the steel pipe 2 was pushed into the plug 1. At this time, the steel pipe 2 was pushed until the plug 1 was passed through the whole length of the steel pipe 2. The pipe expansion ratio was 2.0% in each case.

The 150-t press machine is provided with a load cell, and the working load in the pipe expansion was determined using the load cell.

The pipe expansion work was performed while variously changing the condition of lubricant. The test condition is shown in Table 6.

[Table 6]

TABLE 6

| | Lubricant | |
|---|---|---|
| Test condition | Steel pipe inner surface | Plug surface |
| 1 | Non | Non |
| 2 | Mineral oil | Non |
| 3 | Water | Non |
| 4 | Non | Na stearate (not dried) |
| 5 | Na stearate (not dried) | Na stearate (not dried) |
| 6 | Na stearate (dried) | Non |
| 7 | Na stearate (not dried) | Non |

As shown in Table 6, in Test condition 1, the pipe expansion was carried out without coating any lubricant to the steel inner surface. In test condition 2, the pipe expansion was carried out after coating mineral oil (manufactured by Idemitu Kosan, SD22) to the whole steel pipe inner surface. In Test condition 3, the pipe expansion was carried out after coating water as lubricant to the whole steel pipe inner surface. In Test condition 4, the pipe expansion was carried out after coating Na stearate aqueous solution with concentration of 100 g/L (liter) as lubricant to the plug surface and substantially perfectly drying and solidifying the lubricant by air blowing for 10 minutes. No lubricant was coated to the steel pipe inner surface in Test condition 4. In Test condition 5, the same Na stearate aqueous solution as in Test condition 4 was coated to the whole steel pipe inner surface and to the whole plug surface, thereafter the pipe expansion was performed before the coated Na stearate aqueous solution was dried. In Test condition 6, the pipe expansion was carried out after the same Na stearate aqueous solution as in Test condition 4 was coated to the whole steel pipe inner surface, and dried by air blowing for 10 minutes to form a solid Na stearate layer. In Test condition 7, the same Na stearate aqueous solution in Test condition 4 was coated to the whole steel pipe inner surface, and the pipe expansion was carried out before it is dried. The Na stearate aqueous solution of each condition was prepared within a plastic vessel. In the conditions other than Test conditions 4 and 5, no lubricant was coated to the plug surface.

In each test condition, the pipe expansion was carried out using part or all of the plugs of Plug Nos. 1 to 3.

The test result is shown in FIG. 4. In the drawing, each black bar chart shows the load in pipe expansion using the plug of Plug No. 1. Each white bar chart shows the load in use of the plug of Plug No. 2. Each internally hatched bar chart shows the load in use of the plug of Plug No. 3.

In use of each of the plugs of Plug Nos. 1 to 3, the load was minimized in Test condition 6. Namely, the load in the pipe expansion could be reduced more in Test condition 6, with the Na stearate layer formed on the working surface, than in Test condition 2 using mineral oil as in the conventional pipe expansion work. The load was reduced more in Test condition 6, in which the coated Na stearate was dried, than Test conditions 4 and 7, in which the pipe expansion work was carried out before drying it. This result is attributed to that, since the adhesiveness of Na stearate to the working surface (inner surface) was higher in its dried state, the function of the lubricant was further expressed.

After the pipe expansion work, the inner surface of each steel pipe product, which was expanded in Test conditions 2 and 6, was washed with water. Specifically, a water of normal temperature was injected from a nozzle with an inside diameter of 3.6 mm at a rate of 8 L (liter)/min to wash the steel pipe's inner surface. Consequently, the mineral oil layer coated in Test condition 2 was scarcely removed, while the Na stearate layer coated as the lubricant in Test condition 6 was fully removed.

Table 7 is a result of water washing that was separately carried out at a hydraulic pressure of 5 MPa to the Na stearate layer coated as the lubricant in Test condition 6. The washing removability of Na stearate layer after the pipe expansion work was evaluated by variously changing the time from the pipe expansion work of the steel pipe end using the plug at the start of washing. At this time, the temperature (° C.) of the washing water and the washing time (see) were varied. Consequently, it could be confirmed that the Na stearate layer can be easily removed by water washing regardless of the temperature of washing water (10-80° C.) and the washing time (20-30 sec) if the washing is started within 1 hour after the pipe expansion work.

[Table 7]

TABLE 7

| | Time to start of | | Temperature of | Washing | |
|---|---|---|---|---|---|
| Washing | washing | | washing | time | |
| condition | after working | | water (° C.) | (sec) | Evaluation |
| 1 | 5 | min | 10 | 20 | ◯ |
| 2 | 5 | min | 20 | 20 | ◯ |
| 3 | 5 | min | 30 | 20 | ◯ |
| 4 | 5 | min | 80 | 20 | ◯ |
| 5 | 1 | hour | 10 | 20 | ◯ |
| 6 | 1 | hour | 20 | 20 | ◯ |
| 7 | 1 | hour | 30 | 20 | ◯ |
| 8 | 1 | hour | 80 | 20 | ◯ |
| 9 | 3 | hours | 10 | 20 | X |
| 10 | 3 | hours | 20 | 20 | Δ |
| 11 | 3 | hours | 20 | 30 | ◯ |
| 12 | 3 | hours | 30 | 20 | ◯ |
| 13 | 3 | hours | 80 | 20 | ◯ |

(Note)
Evaluation:
◯: Layer was perfectly removed by washing.
Δ: Layer was almost removed by washing, but partially left.
X: Layer was almost left after washing.

As an additional test, a plurality of alkali soap lubricants, which differed in concentration of Na stearate, was prepared. Specifically, three kinds of alkali soap lubricants of (1) Na stearate aqueous solution having a concentration of 200 g/L, (2) aqueous pasty Na stearate obtained by impregnating Na stearate with hot water of about 80° C. to a concentration of 350 g/L followed by cooling to room temperature, and (3) aqueous pasty Na stearate obtained by impregnating Na stearate with hot water of about 80° C. to a concentration of 450 g/L followed by cooling to room temperature were prepared.

Each of the prepared alkali soap lubricants was coated to the whole inner surface of the above-mentioned steel pipe and dried by air blowing for 10 minutes to form Na stearate layer, thereafter the pipe expansion was carried out. Consequently, in each alkali soap lubricant, the load reducing effect of the same degree as in the Na stearate aqueous solution, with concentration of 100 g/L used in Test condition 6, was obtained.

### EXAMPLE 2

A pipe end of a stainless steel pipe was expanded using Na stearate and conventional mineral oil as lubricants, respectively, and the load applied in pipe expansion was examined for each lubricant.

A super-13Cr steel pipe (hereinafter simply referred to as stainless steel pipe) with an outside diameter 114.3 mm, a thickness 8.56 mm and an inside diameter 97.18 mm was prepared as a steel pipe material.

A plug used for the pipe expansion was made of cemented carbide. This plug has a TD-treated surface and a shape similar to that of FIG. 3. The maximum plug diameter of the plug is 98.15 mm.

The pipe expansion was carried out according to the following method. Ten stainless steel pipes were prepared, in which the inner surface was at least within the range of 50 mm from the pipe end and was coated with Na stearate aqueous solution of 100 g/L (liter) uniformly, and substantially dried. The Na stearate aqueous solution was prepared within a plastic vessel. In order to compare the material, four stainless steel pipes were prepared, in which the inner surface of the pipe end, within the same range as above, was coated with conventional mineral oil.

The pipe end portion of 50 mm in length from the pipe end of each steel pipe was expanded at normal temperature, using hydraulic machining equipment mounted with the above-mentioned plug. The pipe expansion rate was 1.0%. The maximum value and minimum value of the original pressure of the hydraulic machining equipment in pipe expansion were measured. Based on the measurement result, the average values of the maximum value and minimum value of original pressure were determined for each lubricant.

The examination result is shown in FIG. 5. In the drawing, the vertical axis shows the original pressure (kgf/cm²). In the drawing, each white bar chart shows the average of the maximum value of original pressure, and each black bar chart shows the average of the minimum value of original pressure. As is referred from FIG. 5, the maximum value and minimum value of the original pressure were lower in the Na stearate than in the mineral oil.

After the pipe expansion work, the inner surface of each steel pipe was washed in the same condition as in Example 1. Consequently, only a small amount of the mineral oil was removed, while the Na stearate was easily removed without any remaining.

### INDUSTRIAL APPLICABILITY

According to the present invention, a layer of the lubricant can be easily formed on a surface of a steel pipe prior to cold working, in which much reduces the workload during cold working of the steel pipe. A layer of the lubricant can be also easily removed by washing the steel pipe surface after cold working. The present invention is applicable to cold working, particularly, pipe expansion work of the steel pipe end using a plug.

### BRIEF DESCRIPTION OF THE DRAWINGS

FIG. 1 is a flow chart showing each process of a cold working method according to an embodiment of the present invention.

FIG. 2 is a side view showing the shape of a plug used in Example 1.

FIG. 3 is a schematic view of a pipe expansion apparatus used in Example 1.

FIG. 4 is a view showing the pipe expansion load value in each test condition determined in Example 1.

FIG. 5 is a view showing the original pressure value of hydraulic machining equipment in each lubricating treatment determined in Example 2.

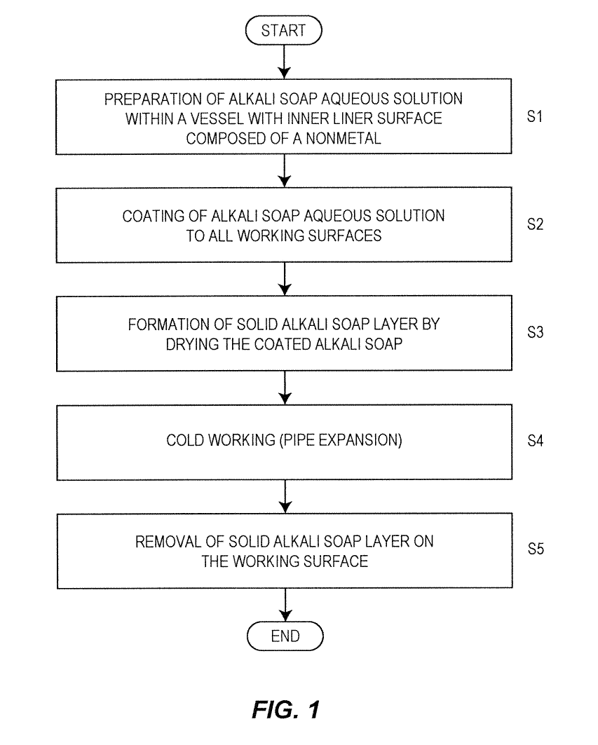

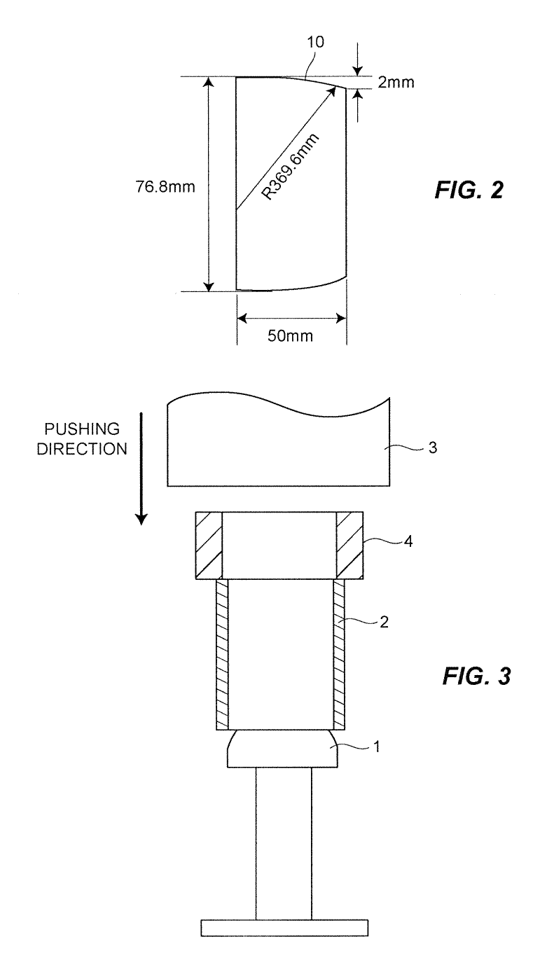

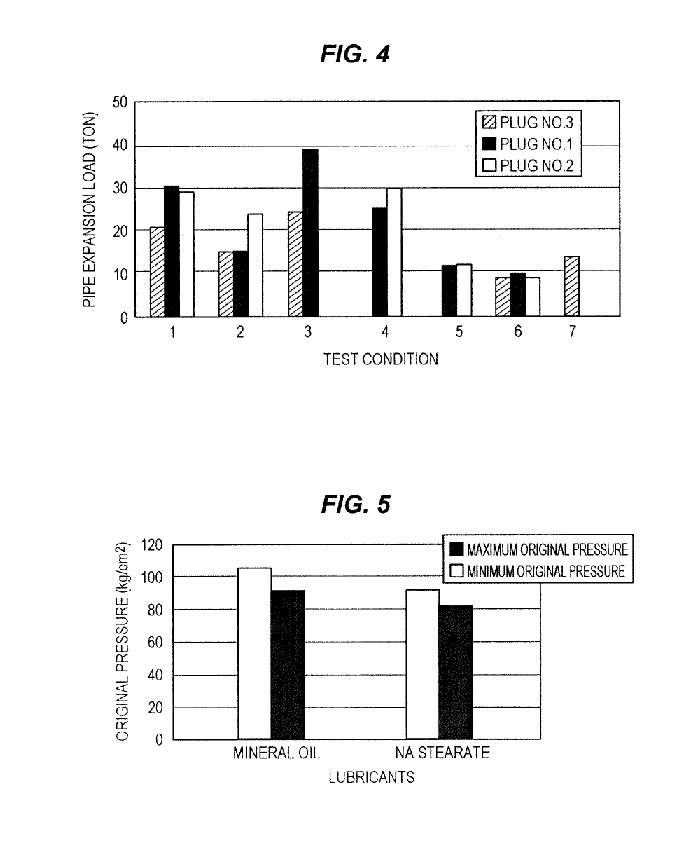

### EXPLANATION OF REFERENCE NUMERALS

1. Plug
2. Steel pipe
3. Press head
4. Pushing and pulling tool

1. A cold working lubricant for a steel pipe, comprising alkali soap.

2. The cold working lubricant for a steel pipe according to claim 1, wherein the cold working of a steel pipe is a pipe expansion work of a steel pipe end using a plug.

3. The cold working lubricant for a steel pipe according to claim 1, wherein the alkali soap is composed of either or both of Na salt and K salt of one or more kinds of straight-chain fatty acids having 10 to 18 carbon atoms.

4. The cold working lubricant for a steel pipe according to claim 1, wherein the cold working of a steel pipe is a pipe expansion work of a steel pipe end using a plug, and wherein the alkali soap is composed of either or both of Na salt and K salt of one or more kinds of straight-chain fatty acids having 10 to 18 carbon atoms.

5. A cold working method for a steel pipe, comprising cold working after forming a solid alkali soap layer on the working surface of a steel pipe by coating an alkali soap aqueous solution thereto.

6. A cold working method for a steel pipe, comprising cold working after forming a solid alkali soap layer on the working surface of a steel pipe by coating an alkali soap aqueous solution thereto followed by drying.

7. The cold working method for a steel pipe according to claim 5, wherein the alkali soap aqueous solution to be coated to the working surface of the steel pipe is prepared by dissolving alkali soap in water within a vessel having an inner surface consisting of a non-metal material.

8. The cold working method for a steel pipe according to claim 6, wherein the alkali soap aqueous solution to be coated to the working surface of the steel pipe is prepared by dissolving alkali soap in water within a vessel having an inner surface consisting of a non-metal material.

9. A cold working method for a steel pipe, comprising cold working after forming a solid alkali soap layer on the working surface of a steel pipe by coating an aqueous pasty alkali soap thereto.

10. A cold working method for steel pipe, comprising cold working after forming a solid alkali soap layer on a working surface of a steel pipe by coating an aqueous pasty alkali soap thereto followed by drying.

11. The cold working method for a steel pipe according to claim 9, wherein the aqueous pasty alkali soap to be coated to the working surface of the steel pipe is prepared by impregnating alkali soap with water within a vessel having an inner surface consisting of a non-metal material.

12. The cold working method for a steel pipe according to claim 10, wherein the aqueous pasty alkali soap to be coated to the working surface of the steel pipe is prepared by impregnating alkali soap with water within a vessel having an inner surface consisting of a non-metal material.

13. The cold working method for a steel pipe according to any one of claims 6 to 12, wherein the cold working of the steel pipe is a pipe expansion work of the steel pipe end using a plug.

14. The cold working method for a steel pipe according to any one of claims 5 to 12, wherein the alkali soap is composed of either or both of Na salt and K salt of one or more kinds of straight-chain fatty acids having 10 to 18 carbon atoms.

15. The cold working method for a steel pipe according to any one of claims 5 to 12, wherein the cold working of the steel pipe is a pipe expansion work of the steel pipe end using a plug, and wherein the alkali soap is composed of either or both of Na salt and K salt of one or more kinds of straight-chain fatty acids having 10 to 18 carbon atoms.
